(19) United States
(12) Patent Application Publication
(10) Pub. No.: US 2021/0088772 A1
(43) Pub. Date: Mar. 25, 2021

(54) ENDOSCOPE APPARATUS, OPERATION METHOD OF ENDOSCOPE APPARATUS, AND INFORMATION STORAGE MEDIA

(71) Applicant: OLYMPUS CORPORATION, Tokyo (JP)

(72) Inventors: Yasunori MORITA, Tokyo (JP); Jumpei TAKAHASHI, Tokyo (JP)

(73) Assignee: OLYMPUS CORPORATION, Tokyo (JP)

(21) Appl. No.: 17/117,584

(22) Filed: Dec. 10, 2020

Related U.S. Application Data
(63) Continuation of application No. PCT/JP2018/023317, filed on Jun. 19, 2018.

(51) Int. Cl. G02B 23/24; A61B 1/04

(52) U.S. Cl. CPC G02B 23/2461; A61B 1/0638; A61B 1/04; G02B 23/2423

(57) ABSTRACT

An endoscope apparatus includes: an illumination device emitting first and second lights; an imaging section capturing an image of return light from a subject based on emission by the illumination device; and a processor including hardware. The first light has a peak wavelength within a first wavelength range including a wavelength at which absorbance of a biological mucosa reaches a largest value. The second light has a peak wavelength within a second wavelength range including a wavelength at which absorbance of a muscle layer reaches a maximum value, and absorbance of the second light by fat is lower than that by the muscle layer. The processor displays the biological mucosa and the muscle layer in an identifiable manner based on a first image corresponding to the first light, and displays the muscle layer and the fat in an identifiable manner based on a second image corresponding to the second light.

### CROSS REFERENCE TO RELATED APPLICATION

This application is a continuation of International Patent Application No. PCT/JP2018/023317, having an international filing date of Jun. 19, 2018, which designated the United States, the entirety of which is incorporated herein by reference.

### BACKGROUND

For in-vivo observation and treatment using an endoscope apparatus, methods for highlighting a specific object through image processing are widely known. For example, Japanese Unexamined Patent Application Publication No. 2016-067775 discloses a method for highlighting information on blood vessels located at a specific depth based on image signals taken by emission of light within a specific wavelength band. International Publication No. WO2013/115323 discloses a method for highlighting a fat layer by emission of illumination light within a plurality of wavelength bands taking into account an absorption characteristic of ß-carotene.

A procedure of transurethrally resecting a bladder tumor using an endoscope apparatus (transurethral resection of the bladder tumor; TUR-Bt) is widely known.

In TUR-Bt, a tumor is resected in the state where the bladder is filled with a perfusion solution. The bladder wall is thinly stretched due to the perfusion solution. As the procedure is done in this state, TUR-Bt involves a risk of perforation. The bladder wall consists of three layers of a mucosa layer, a muscle layer, and a fat layer in this order from inside to outside. Hence, displaying the layers in such a manner as to allow for easy identification of each layer would help avoid perforation.

### SUMMARY

According to one aspect of the disclosure, there is provided an endoscope apparatus including: an illumination device configured to emit a plurality of illumination lights including first light and second light; an imaging device configured to capture an image of return light from a subject based on emission by the illumination device; and a processor including hardware. The illumination device is configured to: emit the first light having a peak wavelength within a first wavelength range including a wavelength at which absorbance of a biological mucosa reaches a largest value; and emit the second light having a peak wavelength within a second wavelength range including a wavelength at which absorbance of a muscle layer reaches a maximum value, absorbance of the second light by fat being lower than absorbance of the second light by the muscle layer. The processor is configured to: generate a display image based on a first image that is captured by the imaging device and that corresponds to the first light to thereby display the biological mucosa and the muscle layer in an identifiable manner; and generate the display image based on a second image that is captured by the imaging device and that corresponds to the second light to thereby display the muscle layer and the fat in an identifiable manner.

According to another aspect of the disclosure, there is provided an operation method of an endoscope apparatus, the method including: emitting a plurality of illumination lights including first light and second light, the first light having a peak wavelength within a first wavelength range including a wavelength at which absorbance of a biological mucosa reaches a largest value, the second light having a peak wavelength within a second wavelength range including a wavelength at which absorbance of a muscle layer reaches a maximum value, absorbance of the second light by fat being lower than absorbance of the second light by the muscle layer; capturing an image of return light from a subject based on emission of the plurality of illumination lights; and performing image processing using a captured first image and a captured second image respectively corresponding to the first light and the second light. In the image processing, the method further includes: generating a display image based on the first image to thereby display the biological mucosa and the muscle layer in an identifiable manner; and generating the display image based on the second image to thereby display the muscle layer and the fat in an identifiable manner.

According to still another aspect of the disclosure, there is provided a non-transitory information storage media storing a program, the program causing a computer to execute steps including: causing an illumination device to emit a plurality of illumination lights including first light and second light, the first light having a peak wavelength within a first wavelength range including a wavelength at which absorbance of a biological mucosa reaches a largest value, the second light having a peak wavelength within a second wavelength range including a wavelength at which absorbance of a muscle layer reaches a maximum value, absorbance of the second light by fat being lower than absorbance of the second light by the muscle layer; capturing an image of return light from a subject based on emission by the illumination device; and performing image processing using a captured first image and a captured second image respectively corresponding to the first light and the second light. At the step of the image processing, the program causes the computer to perform a process of generating a display image based on the first image to thereby display the biological mucosa and the muscle layer in an identifiable manner, and generating the display image based on the second image to thereby display the muscle layer and the fat in an identifiable manner.

### BRIEF DESCRIPTION OF THE DRAWINGS

FIGS. 1A and 1B explain TUR-Bt.

FIG. 2 illustrates a configuration example of an endoscope apparatus.

FIG. 3A illustrates an example of spectral characteristics of illumination light in accordance with an exemplary embodiment, FIG. 3B explains absorbance of a mucosa layer, a muscle layer, and a fat layer, and

FIG. 3C illustrates an example of spectral characteristics of illumination light in white light observation.

FIG. 4 is a flowchart explaining image processing.

FIG. 5 is a schematic diagram explaining a specific procedure of a structure highlighting process.

FIG. 6 is a flowchart explaining the structure highlighting process.

FIG. 7 is a flowchart explaining a color highlighting process.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

### 1. Method of an Exemplary Embodiment

First, a description will be given of a method in accordance with an exemplary embodiment. While the below description takes an example of TUR-Bt, the method of the present embodiment may be applied to other situations that require identification of a mucosa layer, a muscle layer, and a fat layer. In other words, the method of the present embodiment may be applied to other procedures on the bladder, such as transurethral resection of bladder tumor in one-piece (TUR-BO), and may also be applied to observations and procedures on portions other than the bladder.

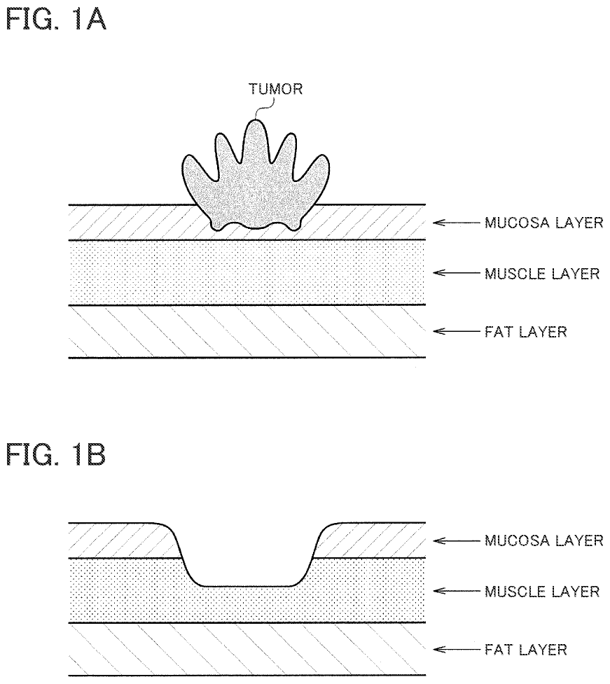

FIGS. 1A and 1B explain TUR-Bt. FIG. 1A schematically illustrates an example of a portion of the bladder wall having a tumor thereon. The bladder wall consists of three layers of a mucosa layer, a muscle layer, and a fat layer, from inside to outside in this order. The tumor stays in the mucosa layer at its relatively early stage, but gradually invades deeper layers including the muscle layer and the fat layer as it develops. By way of example, FIG. 1A illustrates the tumor that has not invaded the muscle layer.

FIG. 1B schematically illustrates an example of a portion of the bladder wall with the tumor resected therefrom by TUR-Bt. In TUR-Bt, at least a portion of the mucosa layer around the tumor is resected. For example, the mucosa layer and a portion of the muscle layer near the mucosa layer are resected. The resected tissue is subject to pathological diagnosis, by which the nature of the tumor and how deep the tumor has grown into the bladder wall are examined. When the tumor is a non-muscle invasive cancer as illustrated in FIG. 1A, the tumor is completely resectable by TUR-Bt, depending on its pathological condition. In other words, TUR-Bt is a procedure that combines diagnosis and treatment.

In view of completely resecting a relatively early stage tumor that has not invaded the muscle layer, resecting the bladder wall up to its relatively deep layer is of importance in the case of TUR-Bt. For example, it is desirable to resect the bladder wall up to an intermediate portion of the muscle layer so that the mucosa layer around the tumor does not remain unremoved. Meanwhile, during TUR-Bt, the bladder wall is being thinly stretched due to a perfusion solution. Hence, resecting the bladder wall excessively up to its deep layer increases a risk of perforation. For example, it is desirable not to resect the fat layer.

To enable an appropriate resection by TUR-Bt, identification of the mucosa layer, the muscle layer, and the fat layer is important. Japanese Unexamined Patent Application Publication No. 2016-067775 is directed to a method for improving visibility of blood vessels, not to a method for highlighting regions respectively corresponding to the mucosa layer, the muscle layer, and the fat layer. International Publication No. WO2013/115323 discloses a method for displaying the fat layer in a highlighting manner, but this method does not individually identify the three layers including the mucosa layer and the muscle layer as well as the fat layer.

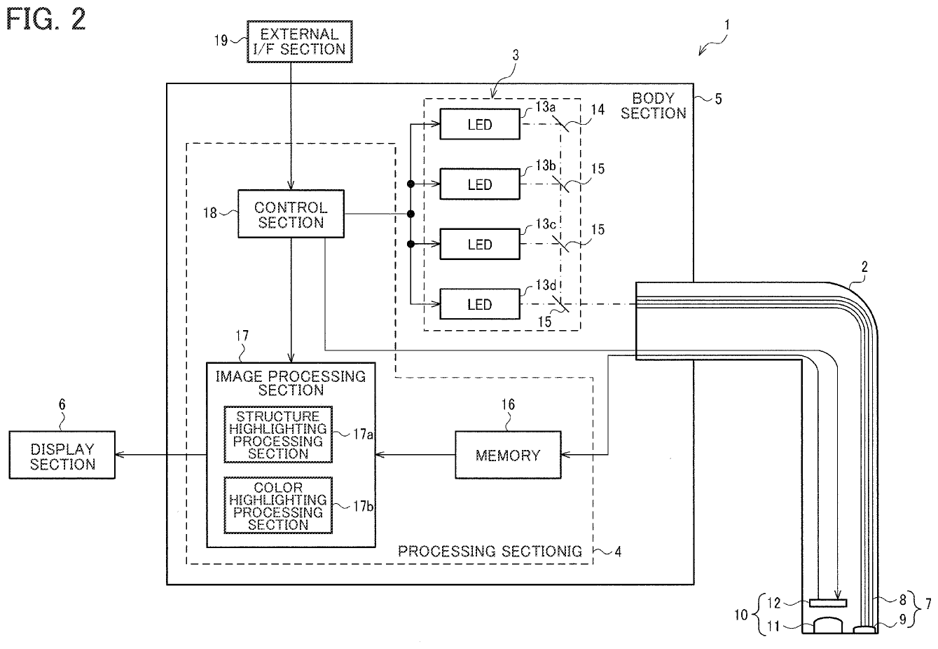

As shown in FIG. 2, an endoscope apparatus 1 in accordance with the present embodiment may include an illumination section 3, an imaging section 10, and an image processing section 17. The illumination section 3 emits a plurality of illumination lights including first light and second light. The imaging section 10 captures images of return light from a subject based on the emission by the illumination section 3. The image processing section 17 performs image processing using a first image and a second image corresponding to the first light and the second light, respectively, captured by the imaging section 10.

The first light and the second light satisfy the following characteristics. The first light has a peak wavelength within a first wavelength range that includes a wavelength at which absorbance of a biological mucosa reaches the largest value. The second light has a peak wavelength within a second wavelength range that includes a wavelength at which absorbance of the muscle layer reaches a maximum value, and absorbance of the second light by fat is lower than absorbance of the second light by the muscle layer. The peak wavelength of the first light refers to the wavelength at which intensity of the first light becomes the largest. This holds for peak wavelengths of other light, and the wavelength at which intensity of the respective light becomes the largest is the peak wavelength of that respective light.

Each of the mucosa layer (biological mucosa) and the muscle layer is an object that contains a large amount of myoglobin. However, the myoglobin concentration is relatively high in the mucosa layer and relatively low in the muscle layer. This difference in concentration leads to difference in absorption characteristics between the mucosa layer and the muscle layer. This absorbance difference becomes the largest near the wavelength at which the absorbance of the biological mucosa reaches the largest value. In other words, the first light produces a large difference between the mucosa layer and the muscle layer as compared to other light having a peak wavelength within other wavelength bands. Specifically, the first image captured by emission of the first light produces a large difference between pixel values of a region capturing the mucosa layer and pixel values of a region capturing the muscle layer, as compared to images captured by emission of other light.

Since absorbance of the second light by fat is lower than that by the muscle layer, pixel values of a region capturing the muscle layer are smaller than pixel values of a region capturing the fat layer in the second image captured by the emission of the second light. In particular, since the second light corresponds to the wavelength at which absorbance of the muscle layer reaches a maximum value, difference between the muscle layer and the fat layer, namely difference between pixel values of the muscle layer region and pixel values of the fat layer region in the second image can be identifiably large.

As described above, the method of the present embodiment allows for identification of the mucosa layer and the muscle layer through the use of the first light and also allows for identification of the muscle layer and the fat layer through the use of the second light. This in turn allows for display of an image captured from an object with the three-layer structure including the mucosa layer, the muscle layer, and the fat layer, in an easily identifiable manner from each other. When applied to TUR-Bt, the method of the present embodiment allows for resection up to an appropriate depth while reducing the risk of perforation. As mentioned, the bladder wall has a three-layer structure of the mucosa layer, the muscle layer, and the fat layer on top of each other in this order from inside to outside. Hence, taking the middle muscle layer as a reference, it is possible to identify between the muscle layer and the mucosa layer and between the muscle layer and the fat layer, thereby identifying the three layers from each other.

### 2. System Configuration Example

FIG. 2 illustrates a system configuration example of the endoscope apparatus 1. The endoscope apparatus 1 includes an insertion section 2, a body section 5, and a display section 6. The body section 5 includes the illumination section 3 connected to the insertion section 2, and a processing section 4.

The insertion section 2 is a portion inserted into a living body. The insertion section 2 includes an illumination optical system 7 that emits light input from the illumination section 3 toward an object, and an imaging section 10 that captures an image of reflected light from the object. Specifically, the imaging section 10 is an imaging optical system.

The illumination optical system 7 includes a light guide cable 8 that guides the light incident from the illumination section 3 to a distal end of the insertion section 2, and an illumination lens 9 that diffuses the light to illuminate the object. The imaging section 10 includes an objective lens 11 that focuses the light emitted by the illumination optical system 7 and reflected by the object, and an image sensor 12 that captures an image of the light focused by the objective lens 11. The image sensor 12 may be implemented by any of various sensors including charge coupled device (CCD) sensors and complementary MOS (CMOS) sensors. Analog signals sequentially output from the image sensor 12 are converted into digital images by an A/D conversion section (not shown). The A/D conversion section may be included either in the image sensor 12 or in the processing section 4.

The illumination section 3 includes a plurality of light emitting diodes (LEDs) 13a-13d each emitting light in a different wavelength band, a mirror 14, and dichroic mirrors 15. Light emitted from each of the plurality of LEDs 13a-13d is made incident into the same light guide cable 8 by the mirror 14 and the dichroic mirrors 15. FIG. 2 illustrates four LEDs, but this is merely exemplary and the number of LEDs is not limited to four. For example, the illumination section 3 may be configured to emit only the first light and the second light, with two LEDs. As another alternative, the illumination section 3 may have three LEDs or five or more LEDs. Details of the illumination light will be given later.

Instead of the LEDs, the light sources of the illumination light may be laser diodes. In particular, the light sources for emitting narrowband light, such as B2 and G2 described later, may be replaced with laser diodes. Still alternatively, the illumination section 3 may sequentially emit light within different wavelength bands by using a white light source for emitting white light, such as a Xenon lamp, and a filter turret including color filters each transmitting a wavelength band corresponding to each illumination light. In this case, the Xenon lamp may be replaced with a combination of a phosphor and a laser diode for exciting the phosphor.

The processing section 4 includes a memory 16, an image processing section 17, and a control section 18. The memory 16 stores image signals acquired by the image sensor 12 for each wavelength of the illumination light. The memory 16 is, for example, a semiconductor memory such as a static random-access memory (SRAM) and a dynamic random-access memory (DRAM), but may also be a magnetic storage device or an optical storage device.

The image processing section 17 performs image processing on the image signals stored in the memory 16. This image processing includes a highlighting process based on the plurality of image signals stored in the memory 16 and a process of generating a combined display image by allocating the image signals to each of a plurality of output channels. The plurality of output channels in this embodiment is comprised of three channels of an R channel, a G channel, and a B channel, but may be alternatively comprised of three channels of a Y channel, a Cr channel, and a Cb channel, or of any other channel configuration.

The image processing section 17 includes a structure highlighting processing section 17a and a color highlighting processing section 17b. The structure highlighting processing section 17a performs a process of highlighting structural information (structural component) in an image. The color highlighting processing section 17b performs a process of highlighting color information. Also, the image processing section 17 generates a display image by allocating image data to each of the plurality of output channels. The display image refers to an image output from the processing section 4 and displayed by the display section 6. The image processing section 17 may perform other processing on the images acquired from the image sensor 12. For example, the image processing section 17 may execute known processing, such as a white balance process and a noise reduction process, as preprocessing or postprocessing for the highlighting processes.

The control section 18 synchronizes the imaging timing of the image sensor 12, the lighting timing of the LEDs 13a-13d, and the image processing timing of the image processing section 17. The control section 18 is a control circuit or a controller, for example.

The display section 6 sequentially displays the display images output from the image processing section 17. In other words, the display section 6 displays a video that consists of the display images as frame images. The display section 6 is a liquid crystal display or an electro-luminescence (EL) display, for example.

An external I/F section 19 is an interface that allows a user to perform an input operation or the like on the endoscope apparatus 1. In other words, the external I/F section 19 may be an interface for operating the endoscope apparatus 1 or an interface for making operational setting for the endoscope apparatus 1. For example, the external I/F section 19 may include a mode switching button for switching observation modes and an adjustment button for adjusting parameters for image processing.

The endoscope apparatus 1 of the present embodiment may be configured as follows. The endoscope apparatus 1 (the processing section 4 in a narrow sense) may include a memory storing information and a processor configured to operate based on the information stored in the memory. The information may include programs and various data, for example. The processor may perform image processing including the highlighting process and controls emission by the illumination section 3.

For example, the processor may implement functions of the respective sections either by individual hardware or integrated hardware. For example, the processor may include hardware, and the hardware may include at least one of a digital signal processing circuit and an analog signal processing circuit. For example, the processor may be composed of one or more circuit devices mounted on a circuit board or may be composed of one or more circuit elements. The circuit device is an integrated circuit (IC), for example. The circuit element is a resistor or a capacitor, for example. The processor may also be a central processing unit (CPU), for example. The processor is, however, not limited to the CPU and may be any of various processors including a graphics processing unit (GPU) and a digital signal processor (DSP). The processor may also be a hardware circuit including an application specific integrated circuit (ASIC). The processor may include an amplifier circuit or a filter circuit that processes analog signals. The memory may be a semiconductor memory such as an SRAM and a DRAM or may be a register. The memory may also be a magnetic storage device such as a hard disk device or an optical storage device such as an optical disc device. For example, the memory stores computer-readable instructions, and functions of the respective sections in the processing section 4 are implemented as the processes by the processor executing the instructions. These instructions may be an instruction set included in a program or may be instructions that cause operations of the hardware circuit included in the processor.

The sections in the processing section 4 of the present embodiment may be implemented as modules of a program running on the processor. For example, the image processing section 17 is implemented as an image processing module. The control section 18 is implemented as a control module configured to perform various controls including synchronization of the emission timing of the illumination light and the imaging timing of the image sensor 12.

The program for implementing the processes performed by the respective sections in the processing section 4 of the present embodiment may be, for example, stored in an information storage device that is a computer-readable medium. For example, the information storage device may be implemented as an optical disk, a memory card, a hard disk drive (HDD), or a semiconductor memory. The semiconductor memory is a read-only memory (ROM), for example. This information storage device may be the memory 16 shown in FIG. 2 or may be one different from the memory 16. The processing section 4 performs various processes in the present embodiment based on the program stored in the information storage device. In other words, the information storage device stores the program for causing a computer to function as each section of the processing section 4. The computer is a device including an input device, a processing section, a storage section, and an output section. The program causes the computer to execute the processing in each section of the processing section 4.

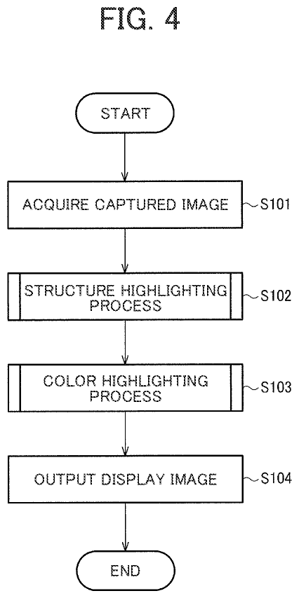

In other words, the method of the present embodiment may be applied to a program that causes a computer to execute steps of causing the illumination section 3 to emit the plurality of illumination lights including the first light and the second light; capturing an image of return light from a subject based on the emission by the illumination section 3; and performing image processing using the captured first image and second image corresponding to the first light and the second light, respectively. The steps executed by the program are those shown in flowcharts of FIGS. 4, 6, and 7. As described above, the first light and the second light have the following characteristics. That is, the first light has a peak wavelength within the first wavelength range that includes a wavelength at which absorbance of a biological mucosa reaches the largest value. The second light has a peak wavelength within the second wavelength range that includes a wavelength at which absorbance of the muscle layer reaches a maximum value, and absorbance of the second light by fat is lower than absorbance of the second light by the muscle layer.

### 3. Details of the Illumination Section

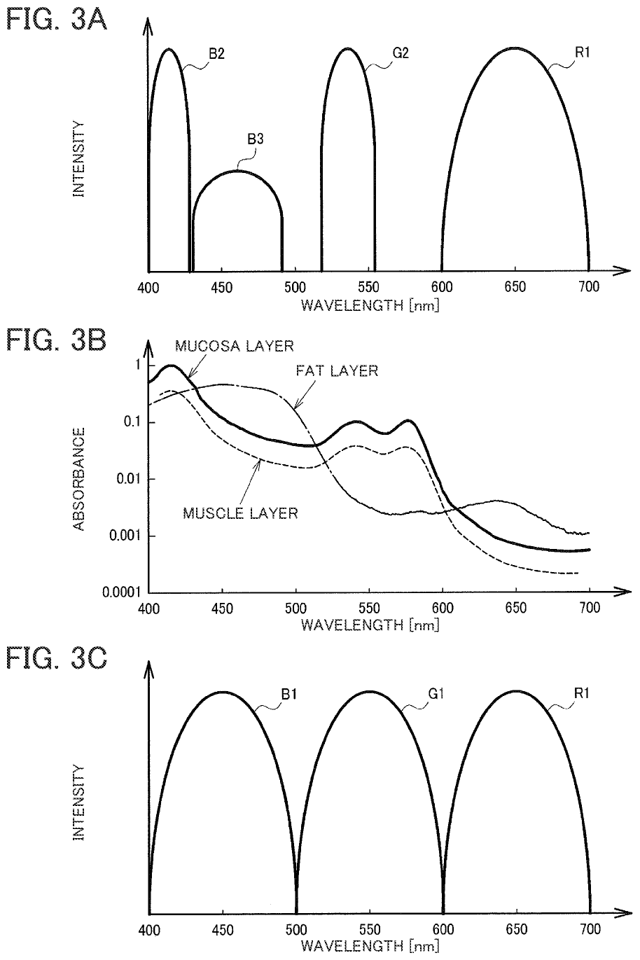

FIG. 3A shows characteristics of the plurality of illumination lights emitted from the plurality of LEDs 13a-13d. In FIG. 3A, the horizontal axis represents wavelength, and the vertical axis represents intensity of the emitted light. The illumination section 3 of the present embodiment includes four LEDs respectively emitting light B2 and B3 within a blue wavelength band, light G2 within a green wavelength band, and light R1 within a red wavelength band.

For example, B2 has a peak wavelength at 415 nm±20 nm. In a narrow sense, B2 is light having intensity at or above a predetermined threshold within a wavelength band of about 400-430 nm. B3 has a longer peak wavelength than B2, and has intensity at or above a predetermined threshold within, for example, a wavelength band of about 430-500 nm. G2 has a peak wavelength at 540 nm±10 nm. In a narrow sense, G2 is light having intensity at or above a predetermined threshold within a wavelength band of about 520-560 nm. R1 has intensity at or above a predetermined threshold within a wavelength band of about 600-700 nm.

FIG. 3B shows absorption characteristics of the mucosa layer, the muscle layer, and the fat layer. In FIG. 3B, the horizontal axis represents wavelength, and the vertical axis represents logarithm of the absorbance. Each of the mucosa layer (biological mucosa) and the muscle layer is an object that contains a large amount of myoglobin. However, the myoglobin concentration is relatively high in the mucosa layer and relatively low in the muscle layer. This difference in concentration leads to difference in absorption characteristics between the mucosa layer and the muscle layer. As shown in FIG. 3B, the absorbance difference is largest near 415 nm at which the absorbance of the biological mucosa becomes the largest. Absorbance of the muscle layer takes a plurality of maximum values. The wavelengths corresponding the respective maximum values are those near 415 nm, 540 nm, and 580 nm.

The fat layer is an object containing a large amount of ß-carotene. In terms of absorption characteristic, the absorbance of ß-carotene significantly drops in a wavelength band of 500-530 nm and becomes flat in a wavelength band above 530 nm. As shown in FIG. 3B, the absorption characteristic of the fat layer is associated with ß-carotene.

As can be seen in FIGS. 3A and 3B, the light B2 has a peak wavelength corresponding to the wavelength at which absorbance of a biological mucosa reaches the largest value. The light G2 corresponds to the wavelength at which absorbance of the muscle layer reaches a maximum value and absorbance of G2 by fat is lower than that by the muscle layer. In the present embodiment, the first light corresponds to B2, and the second light corresponds to G2. The first wavelength range including the peak wavelength of the first light is 415 nm±20 nm. The second wavelength range including the peak wavelength of the second light is 540 nm±10 nm.

When B2 and G2 are set to the wavelengths shown in FIG. 3A, the difference in absorbance between the mucosa layer and the muscle layer within the wavelength band of the light B2 is large enough to identify the two objects from each other. Specifically, in a B2 image obtained by emission of the light B2, a region capturing the mucosa layer has lower pixel values and thus is darker than a region capturing the muscle layer. In other words, a display image generated by using the B2 image can facilitate identification between the mucosa layer and the muscle layer. For example, when the B2 image is allocated to the B output channel, the mucosa layer is displayed in a color with a low contribution of blue while the muscle layer is displayed in a color with a high contribution of blue.

Also, the difference in absorbance between the muscle layer and the fat layer within the wavelength band of the light G2 is large enough to identify the two objects from each other. Specifically, in a G2 image obtained by emission of the light G2, a region capturing the muscle layer has lower pixel values and thus is darker than a region capturing the fat layer. In other words, a display image generated by using the G2 image can facilitate identification between the muscle layer and the fat layer. For example, when the G2 image is allocated to the G output channel, the muscle layer is displayed in a color with a low contribution of green while the fat layer is displayed in a color with a high contribution of green.

FIG. 3C shows characteristics of three illumination lights B1, G1, and R1 used for displaying commonly used white light images. In FIG. 3C, the horizontal axis represents wavelength, and the vertical axis represents intensity of the emitted light. The light B1 corresponds to a blue wavelength band and is within a wavelength band of 400-500 nm for example. The light G1 corresponds to a green wavelength band and is within a wavelength band of 500-600 nm for example. The light R1 corresponds to a red wavelength band and is within a wavelength band of 600-700 nm for example, similarly to FIG. 3A.

As can be seen in FIGS. 3B and 3C, the fat layer, which shows high absorbance of B1 and low absorbance of G1 and R1, is displayed in a yellowish color. The mucosa layer, which shows high absorbance of B1 and G1 and low absorbance of R1, is displayed in a reddish color. The muscle layer, which shows a flatter absorption characteristic than the mucosa layer, is displayed in a whitish color. The term “flat absorption characteristic” means the change in absorbance is small in relation to the change in wavelength. As such, in a white light observation mode, the mucosa layer, the muscle layer, and the fat layer are also displayed in somewhat different colors from each other.

The first light and the second light in accordance with the present embodiment improve color separation of the mucosa layer, the muscle layer, and the fat layer, as compared to the white light observation mode. Hence, for B2 that is the light within the wavelength band corresponding to blue, it is desirable that a wavelength band with a large difference in absorption characteristics between the mucosa layer and the muscle layer has a high contribution and that a wavelength band with a small difference in absorption characteristics between the mucosa layer and the muscle layer has a low contribution. Specifically, the wavelength band of B2 includes a wavelength band near 415 nm, in which the difference in absorption characteristics between the mucosa layer and the muscle layer is large, and does not include a wavelength band of 450-500 nm, in which the difference in absorption characteristics between the mucosa layer and the muscle layer is small. In other words, B2 is narrowband light whose wavelength band is narrower than the light (B1) used in a white light observation mode. For example, B2 has a half-value width of several nanometers to several tens of nanometers. This enables the image processing section 17 to generate a display image in which a color difference between the mucosa layer and the muscle layer is highlighted, as compared to a white light observation mode using the illumination light as shown in FIG. 3C.

Likewise, G2 is within a part of the wavelength band corresponding to green where the difference in absorption characteristics between the muscle layer and the fat layer is large. In other words, G2 is narrowband light whose wavelength band is narrower than the light (G1) used in a white light observation mode. For example, G2 has a half-value width of several nanometers to several tens of nanometers. This enables the image processing section 17 to generate a display image in which a color difference between the muscle layer and the fat layer is highlighted, as compared to a white light observation mode using the illumination light as shown in FIG. 3C.

In terms of displaying the three layers including the mucosa layer, the muscle layer, and the fat layer in an easily identifiable manner, the illumination section 3 is only required to emit the first light B2 and the second light G2. This means that the illumination section 3 of the present embodiment may be configured, for example, to emit the first light B2 and the second light G2 and not to emit any other light.

The illumination section 3 is, however, not limited to this configuration and may be configured to emit light different from both of the first light and the second light. For example, the illumination section 3 emits third light in a wavelength band in which absorbance of the third light by a biological mucosa is lower than absorbance of the first light by the biological mucosa and in which absorbance of the third light by the muscle layer is lower than absorbance of the second light by the muscle layer. As can be seen in FIGS. 3A and 3B, the third light corresponds to R1, for example.

As described above, in the B2 image, the region capturing the mucosa layer is considered to have lower pixel values than the region capturing the muscle layer. It should be noted that brightness of an object on an image varies depending on positional relationship between the object and the insertion section 2. For example, an object at a relatively close distance from the insertion section 2 is captured brightly as compared to another object relatively distant from the insertion section 2. Also, if an object has an uneven surface, a convex portion of the surface may block the illumination light and/or the reflection light from the object, so that a region around the convex portion may be captured darkly as compared to other regions. In TUR-Bt, the convex portion is a tumor, for example.

As such, brightness of an object, i.e., pixel values of the object, may change depending on how easily the illumination light can reach the object or how easily the reflection light therefrom can be received. If a region where pixel values of the mucosa layer are high, which would be normally low, or a region where pixel values of the muscle layer are low, which would be normally high, occurs in the B2 image, such a region may reduce color separability between the mucosa layer and the muscle layer using the B2 image. In this regard, the mucosa layer has low absorbance of R1. As compared to the B2 image, an R1 image captured by emission of R1 produces less difference in pixel values associated with the difference in absorption characteristics between the mucosa layer and the muscle layer. Hence, combined use of the first light and the third light can improve color separability between the mucosa layer and the muscle layer. A specific example of the image processing will be described later.

Similarly, absorbance of the third light R1 by the muscle layer is lower than absorbance of the second light by the muscle layer. Thus, as compared to the G2 image, the R1 image produces a smaller difference in pixel values associated with the difference in absorption characteristics between the muscle layer and the fat layer. Hence, combined use of the second light and the third light can improve color separability between the muscle layer and the fat layer. A specific example of image processing in this case will also be described later.

The light R1 as the third light also allows for improving color rendering properties of the display image. If the display image is generated by use of the first light and the second light alone, that display image is a pseudo-color image. In this regard, adding R1 as the third light allows for emission of light in the blue wavelength band (B2), light in the green wavelength band (G2), and light in the red wavelength band (R1). Then, the B2 image is allocated to the B output channel. The G2 image is allocated to the G output channel. The R1 image is allocated to the R output channel. Eventually, the colors of the display image can be closer to the natural colors of a white light image.

Also, the present embodiment does not preclude the illumination section 3 from emitting fourth light that is different from any of the first to third lights. The fourth light is B3 shown in FIG. 3A, for example. As the first light B2 is narrowband light in a narrow sense, the B2 image is apt to be dark as compared to a B1 image that is captured in a white light observation mode. Hence, the use of the B2 image for generation of the display image may reduce visibility of an object therein. Besides, any correction process on the B2 image in an attempt to increase its pixel values may increase noise.

In this regard, emission of B3 as the fourth light by the illumination section 3 can appropriately enhance brightness of the image corresponding to the blue wavelength band. For example, the illumination section 3 emits B2 and B3 simultaneously, and the imaging section 10 receives the reflection light resulting from emission of these two lights. Alternatively, the illumination section 3 emits B2 and B3 at different timings, and the image processing section 17 combines the B3 image captured by emission of B3 with the B2 image and thereafter allocates the combined image to the B output channel. The image processing section 17 can generate a bright display image that ensures high visibility of objects therein.

However, when the illumination section 3 is caused to emit B3, intensities of B2 and B3 need to be carefully set in a proper relationship. This is because emission of B3 is intended to enhance the intensity of light in the blue wavelength band, with no consideration for identification between the mucosa layer and the muscle layer. That is, if the intensity of B3 is too high, the combined emission of B2 and B3 makes identification between the mucosa layer and the muscle layer difficult, as compared to the emission of B2 alone. For example, if B2 and B3 have a similar intensity, color separability would be comparable to that of a normal white light observation mode using B1, which may impair the advantages of using B2.

Hence, the intensity of B2 is set higher than that of B3. Preferably, the intensity of B2 is set higher than that of B3 to the extent that the contribution of B2 is dominant in the blue wavelength band. The intensity of the illumination light is controlled, for example, by the amount of electric current supplied to the LEDs. Setting the intensity in this way allows for displaying the mucosa layer and the muscle layer in an easily identifiable manner and also allows for generating a bright display image.

The foregoing description has taken B2 as the first light, and B3 as the fourth light that is different from the first light. The illumination section 3 may, instead, emit a single light combining the characteristics of B2 and B3 as the first light in accordance with the present embodiment. For example, the illumination section 3 may emit the first light having the combined characteristics of B2 and B3 by simultaneously lighting the LED corresponding to B2 and the LED emitting B3. Alternatively, the illumination section 3 may emit the first light having combined characteristics of B2 and B3 by combining a light source that emits light containing at least the blue wavelength band, such as a white light source, and a filter.

In this case, the first light has a wavelength whose transmittance is maximized at 415 nm±20 nm and has such a characteristic that transmittance at the wavelength near 415 nm is distinguishably higher than that in a wavelength band above 430 nm. It is thereby possible to emit blue illumination light whose wavelength band is as wide as B1, and also to make the effect of the wavelength band near 415 nm dominant in this illumination light. In other words, the light that ensures both brightness and color separability can be emitted as the first light.

As described above, the first light in accordance with the present embodiment may be narrowband light (B2) whose wavelength band is narrower than that of the light used in a white light observation mode or may be broad illumination light.

While the description has been given of the light of the blue wavelength band, the same holds for the green wavelength band. That is, the illumination section 3 may emit G2 as the second light, and may also emit G3 (not shown) that has lower intensity than G2 and that corresponds to the green wavelength band. It is thereby possible to display the muscle layer and the fat layer in an easily identifiable manner, and also to generate a bright display image.

Further, the second light is not limited to narrowband light, and the illumination section 3 may emit a single light combining the characteristics of G2 and G3 as the second light in accordance with the present embodiment.

As described above with reference to FIG. 3B, absorbance of the muscle layer reaches a maximum value also at 580 nm, where absorbance of the fat layer is substantially lower than that of the muscle layer. That is, the second light in accordance with the present embodiment is not limited to the light having a peak wavelength at 540 nm±10 nm and may be light having a peak wavelength at 580 nm±10 nm.

While FIG. 3A shows the four illumination lights of B2, B3, G2, and R1 by way of example, the illumination section 3 may emit other light. For example, it is possible to add aforementioned G3 or red narrowband light (not shown).

The endoscope apparatus 1 in accordance with the present embodiment may be able to switch between a white light observation mode and a special light observation mode. In the white light observation mode, the illumination section 3 emits B1, G1, and R1 shown in FIG. 3C. In the special light observation mode, the illumination section 3 emits illumination light including the first light and the second light. For example, in the special light observation mode, the illumination section 3 emits B2, B3, G2, and R1 shown in FIG. 3A. Note that the switch between the observation modes is made through the external I/F section 19, for example.

### 4. Details of Image Processing

Now a description will be given of image processing performed by the image processing section 17.

4.1 Overall Processing

FIG. 4 is a flowchart explaining the processing by the image processing section 17 of the present embodiment. At the start of this processing, the image processing section 17 acquires images captured by the image sensor 12 (S101). As the processing at S101, the image processing section 17 may acquire digital data having undergone A/D conversion by the A/D conversion section included in the image sensor 12, or the image processing section 17 may convert analog signals output from the image sensor 12 into digital data.

Based on the acquired image, the image processing section 17 performs a structure highlighting process (S102) and a color highlighting process (S103). The image processing section 17 then outputs a display image in which data including the images having undergone the above highlighting processes is allocated to each of the plurality of output channels (S104). In the example shown in FIG. 2, the display image is output to the display section 6, which in turn displays the display image.

While the color highlighting process follows the structure highlighting process in FIG. 4, the order of the processes is not limited to this. That is, the structure highlighting process may follow the color highlighting process, or alternatively the structure highlighting process and the color highlighting process may be performed in parallel. Still alternatively, the image processing section 17 may omit a part or all of the highlighting processes at S102 and S103.

In the case where the illumination section 3 emits the first light (B2) and the second light (G2), the image processing section 17 acquires a B2 image and a G2 image at S101, and allocates the B2 image and the G2 image to the respective RGB channels. For example, the image processing section 17 allocates the B2 image to the B and G output channels and the G2 image to the R output channel to thereby generate a display image. The resulting display image is a pseudo-color image which shows, without any highlighting process, the mucosa layer in a brownish color, the muscle layer in a whitish color, and the fat layer in a reddish color. However, the correspondence between the B2 and G2 images and the three output channels is not limited to the above and may be modified in various ways.

In the case where the illumination section 3 emits the first light (B2), the second light (G2), and the third light (R1), the image processing section 17 acquires a B2 image, a G2 image, and an R1 image, and allocates the B2 image to the B output channel, the G2 image to the G output channel, and the R1 image to the R output channel to thereby generate a display image. The resulting display image is close to a white light image and shows, without any highlighting process, the mucosa layer in a reddish color, the muscle layer in a whitish color, and the fat layer in a yellowish color.

In the case where the image sensor 12 includes a color filter, the illumination section 3 simultaneously emits a plurality of lights and the image sensor 12 simultaneously captures a plurality of images. The color filter may be any of a commonly known Bayer array filter, a filter having RGB filters arranged in any other form of array, or a complementary filter. In the case where the illumination section 3 emits the three lights of B2, G2, and R1, the image sensor 12 captures a B2 image based on pixels corresponding to the B filter, a G2 image based on pixels corresponding to the G filter, and an R1 image based on pixels corresponding to the R filter.

The image processing section 17 performs an interpolation process on the output from the image sensor 12 to thereby acquire the B2, G2, and R1 images having signal values in all pixels. In this case, all images are acquired at a time, so that the image processing section 17 can select any of the B2, G2, and R1 images as a target for a highlighting process. Additionally, all signal values of the three output channels are updated in the same frame.

In the case where the image sensor 12 includes multiple monochrome sensors, it is also possible, in one frame, to acquire a plurality of images, perform a highlighting process on the plurality of images, and update signal values of the plurality of output channels.

Meanwhile, in the case where the image sensor 12 includes a single monochrome sensor, it is assumed that one illumination light is emitted and one image corresponding to that illumination light is acquired per frame. When the illumination section 3 emits the three lights of B2, G2, and R1, one period consists of three frames, and a B2 image, a G2 image, and an R1 image are sequentially acquired in that one period. Note that the order of emission of the three illumination lights may be modified in various ways.

The image processing section 17 may acquire all of the B2 image, the G2 image, and the R1 image over three frames at step 101 before going to the processing at S102 and subsequent steps. In this case, the output rate of the display image is ⅓ of the imaging rate. Alternatively, the image processing section 17 may go to the processing at S102 and subsequent steps upon acquiring one of the B2 image, the G2 image, and the R1 image. For example, the image processing section 17 performs a necessary process(es) out of the structure highlighting process and the color highlighting process on the acquired image and allocates that image having undergone the process(es) to any output channel to thereby update the display image. In this case, the output rate of the display image is equal to the imaging rate.

4.2 Structure Highlighting Process

Now a description will be given of the structure highlighting process performed at S102. The image processing section 17 (the structure highlighting processing section 17a) performs at least one of a process of highlighting a structural component of the first image and a process of highlighting a structural component of the second image. The structural component of the first image refers to information representing a structure of an object captured in the first image. For example, the structural component is a specific spatial frequency component.

The structure highlighting processing section 17a extracts a structural component of the first image by filtering the first image. The filter to be applied here may be a bandpass filter that passes a spatial frequency corresponding the structure of an object to be extracted or may be any other edge extracting filter. The processing for extracting the structural component is not limited to filtering and may be any other image processing. The structure highlighting processing section 17a combines the extracted structural component of the first image into the original first image to thereby highlight the structural component of the first image. This combining process may be either a simple addition of the structural component, or determination of a highlighting parameter based on the structural component and subsequent addition of the highlighting parameter. As an alternative, the structure highlighting processing section 17a may extract a plurality of frequency band components by using a plurality of bandpass filters having different passbands. In this case, the structure highlighting processing section 17a highlights the structural component of the first image by a weighted addition of the respective frequency band components. The same applies to a process of highlighting a structural component of the second image.

The above-described highlighting of the structural component causes variation in pixel values of a portion corresponding to the structure, and hence emphasizes the difference in color between the mucosa layer and the muscle layer or between the muscle layer and the fat layer.

The illumination section 3 may also emit the third light whose absorbance by a biological mucosa is lower than absorbance of the first light by the biological mucosa and whose absorbance by the muscle layer is lower than absorbance of the second light by the muscle layer. As described above, the third light is R1, for example. In this case, the structure highlighting processing section 17a corrects the first image based on a third image captured by emission of the third light and highlights the structural component of the corrected first image. Alternatively, the structure highlighting processing section 17a corrects the second image based on the third image and highlights the structural component of the corrected second image. Still alternatively, the structure highlighting processing section 17a performs both of the two correct-and-highlight processes.

The correct-and-highlight process(es) can reduce the influence of uneven brightness due to positional relationship between the object and the insertion section 2 and other factors, and can improve color separability between the mucosa layer and the muscle layer and between the muscle layer and the fat layer. Here, correction of the first image based on the third image is a process of normalizing the first image using the third image, and correction of the second image based on the third image is a process of normalizing the second image using the third image. For example, the structure highlighting processing section 17a performs a correction process based on the third image, using the following expressions (1) and (2).

B2′(x,y)=k1×B2(x,y)/R1(x,y) (1)

G2′(x,y)=k2×G2(x,y)/R1(x,y) (2)

In the above expressions, (x, y) represents a position in the image. B2(x, y) represents a pixel value at (x, y) in the B2 image before the normalization process. Likewise, G2(x, y) represents a pixel value at (x, y) in the G2 image before the normalization process. R1(x, y) represents a pixel value at (x, y) in the R1 image. B2′(x, y) and G2′(x, y) represent pixel values at (x, y) in the B2 image and the G2 image, respectively, after the normalization process. k1 and k2 are given constants. When R1(x, y)=0, B2′(x, y) and G2′(x, y) are 0.

When the R1 image is used for the normalization process, it is desirable that pixel values (luminance values) of the R1 image have an appropriate luminance distribution in accordance with changes in the imaging distance. Hence, the normalization process by the above expressions (1) and (2) may be performed using the R1 image after the correction process, instead of the R1 image itself. For example, a method is known that detects a motion component using a previously captured image and thereby reduces effects coming from receiving regular reflection light. The structure highlighting processing section 17a of the present embodiment may correct the R1 image by this known method. Alternatively, the structure highlighting processing section 17a may perform a noise reduction process, such as a low-pass filter process, on the R1 image, and may perform the normalization process by using the R1 image after the noise reduction process.

While the above expressions (1) and (2) are given as an example of performing the normalization process per pixel, the normalization process may be performed per region composed of a plurality of pixels.

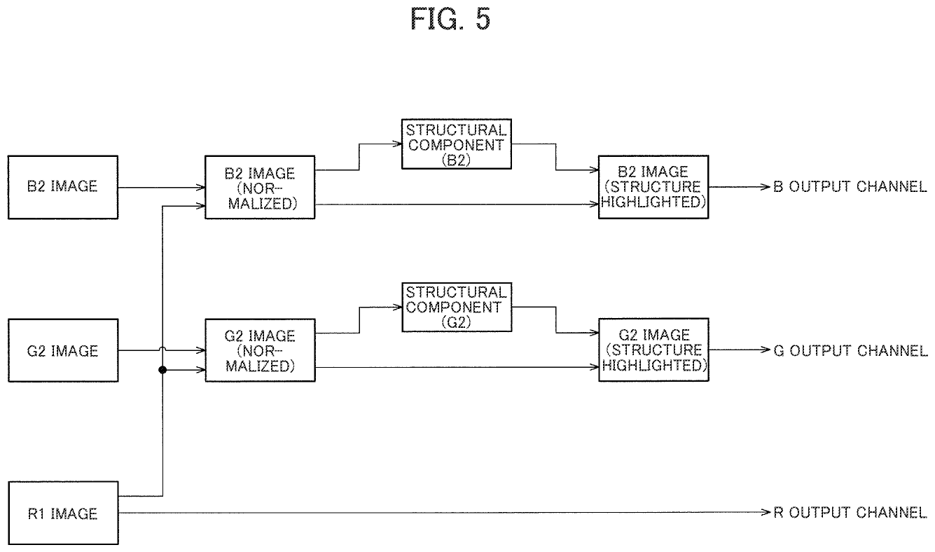

FIG. 5 is a schematic diagram illustrating the procedure of the structure highlighting process described above. As shown in FIG. 5, the normalized B2 image is acquired based on the B2 image and the R1 image, and the normalized G2 image is acquired based on the G2 image and the R1 image. The normalized B2 image is subjected to the filtering, whereby the structural component of the B2 image is extracted. The normalized G2 image is subjected to the filtering, whereby the structural component of the G2 image is extracted. The extracted structural component of the B2 image is combined into the original, normalized B2 image. This combining process produces the B2 image with the highlighted structural component. Likewise, the extracted structural component of the G2 image is combined into the original, normalized G2 image. This combining process produces the G2 image with the highlighted structural component.

Then, the B2 image with the highlighted structural component is allocated to the B output channel. The G2 image with the highlighted structural component is allocated to the G output channel. The R1 image is allocated to the R output channel. A display image is generated through these processes. Eventually, the images with the highlighted structural components are allocated to any of the output channels, which can improve color separability of the mucosa layer, the muscle layer, and the fat layer in the display image.

However, in the endoscope apparatus 1, it is actually the luminance component that makes a structure or motion of an object more recognizable to humans. The luminance component represents a channel, out of the plurality of channels constituting the display image, that is more influential on the luminance of the display image than the other channels. Specifically, the channel more influential on luminance is the G channel. Taking an R signal value, a G signal value, and a B signal value as R, G, and B, respectively, a luminance value Y can be obtained, for example, by the expression Y=r×R+g×G+b×B. Various methods are known for conversion between RGB and YCrCb, and values of the coefficients r, g, and b vary with the method used. In any of the methods, however, g is larger than r, and g is also larger than b. This means that the G signal has a relatively larger contribution to the luminance value Y than the R signal and the B signal.

In the above example, the G2 image after the structure highlighting process is allocated to the G output channel, which corresponds to the luminance component. This allows for display of the muscle layer and the fat layer in an easily identifiable manner to users. On the other hand, the B2 image after the structure highlighting process is allocated to the B channel, which has a lower contribution to the luminance. For this reason, information associated with the B2 image may be less recognizable to users, so that the color separability between the mucosa layer and the muscle layer may be insufficient.

Thus, the image processing section 17 (the structure highlighting processing section 17a) combines the signal corresponding to the structural component into the luminance component in the output. In the above example, the structure highlighting processing section 17a not only combines the structural component of the G2 image extracted from the G2 image into the original G2 image, but also combines the structural component of the B2 image extracted from the B2 image into the original G2 image. This process adds the structural component of the B2 image to the channel that is easily recognizable to users, and thereby allows for display of the mucosa layer and the muscle layer in an easily identifiable manner to users.

In a broad sense, the image processing section 17 (the structure highlighting processing section 17a) combines the signal corresponding to the structural component into at least one of the R, G, and B output signals. Here, the R output signal refers to an image signal allocated to the R output channel. Likewise, the G output signal refers to an image signal allocated to the G output channel, and the B output signal refers to an image signal allocated to the B output channel.

As described above, combining the structural component into the G signal, which corresponds to the luminance component, facilitates recognition by users. In the above example, the G signal corresponds to the G2 image. Combining the structural component of the B2 image into the G signal allows for display of the mucosa layer and the muscle layer in an easily identifiable manner to users. Combining the structural component of the G2 image into the G signal allows for display of the muscle layer and the fat layer in an easily identifiable manner to users.

It should be noted, however, that both the B and R output channels are also components constituting the display image, though their contributions to the luminance is less than the G channel. Thus, combining the structural component into the B component or the R component would also be beneficial to identification of the mucosa layer, the muscle layer, and the fat layer. For example, the structure highlighting processing section 17a may combine the structural component extracted from the B2 image into the R1 image and then allocate the combined image to the R output channel.

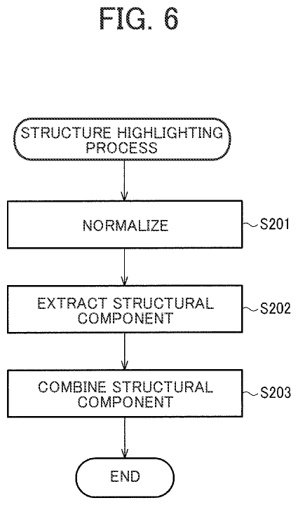

FIG. 6 is a flowchart explaining the structure highlighting process. At the start of this process, the structure highlighting processing section 17a performs the normalization process (S201). Specifically, the structure highlighting processing section 17a calculates the above expressions (1) and (2). Then, the structure highlighting processing section 17a extracts the structural components from the normalized B2 and G2 images (S202). The process at S202 is, for example, application of a bandpass filter. The structure highlighting processing section 17a then combines the structural component extracted at S202 into the signal of at least one of the plurality of output channels (S203). The structural component is combined, for example, into the signal of the G channel corresponding to the luminance component, but may alternatively be combined into the signal of any other channel, as described above.

4.3 Color Highlighting Process

The image processing section 17 (the color highlighting processing section 17b) may also perform a process of highlighting color information based on the captured images. The captured images include the first image, the second image, and the third image. The color information as referred to herein is chroma in a narrow sense, but does not preclude hue or brightness.

Specifically, the image processing section 17 (the color highlighting processing section 17b) performs at least one of a first color highlighting process and a second color highlighting process based on the captured images. The first color highlighting process highlights chroma of a region that is determined as a yellow region. The second color highlighting process highlights chroma of a region that is determined as a red region. In this context, the present embodiment assumes that the illumination section 3 emits the third light (R1) besides the first light (B2) and the second light (G2). Hence, the yellow region is a region corresponding to the fat layer, and the red region is a region corresponding to the mucosa layer.

For example, the color highlighting processing section 17b converts signal values of the respective RGB output channels into luminance Y and color difference components Cr and Cb. Then, the color highlighting processing section 17b detects the yellow region and the red region based on the color difference components Cr, Cb. Specifically, at the color highlighting processing section 17b, a region in a predetermined range in which values of Cr and Cb correspond to yellow is determined as the yellow region, and a region in a predetermined range in which values of Cr and Cb correspond to red is determined as the red region. On at least one of the region determined as the yellow region and the region determined as the red region, the color highlighting processing section 17b performs a chroma highlighting process of increasing chroma values.

The muscle layer has lower chroma than the mucosa layer and the fat layer, and is displayed in a whitish color. Thus, the first color highlighting process to increase the chroma of the fat layer can facilitate identification between the muscle layer and the fat layer. Also, the second color highlighting process to increase the chroma of the mucosa layer can facilitate identification between the mucosa layer and the muscle layer. For improved color separability of the three layers, it is preferable to perform both of the first and second color highlighting processes. However, due to the fact that color separability between two layers is improved by only one of these color highlighting processes, the present embodiment does not preclude omission of the other color highlighting processes. For example, when identification of the fat layer is a matter of high priority in order to reduce the risk of perforation, it is acceptable to perform only the first color highlighting process and to omit the second color highlighting process.

While the description has been given of converting the RGB signals into YCrCb, the color highlighting processing section 17b may instead convert the RGB signals into hue H, chroma (i.e. saturation) S, and brightness (i.e. value) V. In this case, the color highlighting processing section 17b detects the yellow region and the red region based on the hue H, and performs the first color highlighting process and the second color highlighting process by changing values of the chroma (i.e. saturation) S.

The image processing section 17 (the color highlighting processing section 17b) may also perform a third color highlighting process based on the first image, the second image, and the third image. The third color highlighting process is a process of reducing the chroma of a region where the chroma is determined to be below a predetermined threshold. The region where the chroma is below a predetermined threshold corresponds to the muscle layer.

Combination of the first color highlighting process and the third color highlighting process increases the chroma of the fat layer and reduces the chroma of the muscle layer, which in turn increases the difference in chroma between the two layers and facilitates identification between the muscle layer and the fat layer to a further extent. Likewise, combination of the second color highlighting process and the third color highlighting process increases the chroma of the mucosa layer and reduces the chroma of the muscle layer, which in turn increases the difference in chroma between the two layers and facilitates identification between the mucosa layer and the muscle layer to a further extent. Alternatively, the image processing section 17 may perform the third color highlighting process while omitting the first and second color highlighting processes.

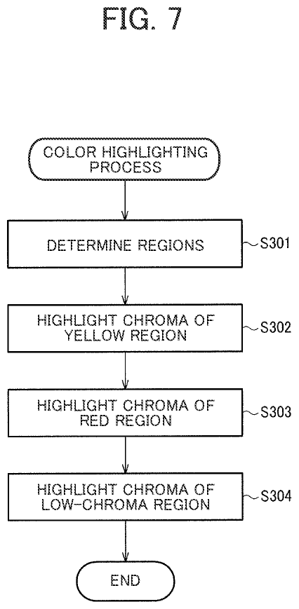

FIG. 7 is a flowchart explaining the color highlighting process. At the start of this process, the color highlighting processing section 17b determines regions in the display image (S301). Specifically, the color highlighting processing section 17b converts the RGB output signals into YCrCb or HSV to thereby detect the yellow region corresponding to the fat layer, the red region corresponding to the mucosa layer, and the low-chroma region corresponding to the muscle layer. In the case of performing the color highlighting process after the structure highlighting process explained with reference to FIG. 5, the R output signal corresponds to the R1 image, the G output signal corresponds to the G2 image with the highlighted structural component, and the B output signal corresponds to the B2 image with the highlighted structural component.

The color highlighting processing section 17b then performs the process of highlighting the chroma of the yellow region (S302), the process of highlighting the chroma of the red region (S303), and the process of highlighting the chroma of the low-chroma region (S304). The chroma highlighting at S302 and S303 is a process of increasing the chroma, whereas the chroma highlighting at S304 is a process of reducing the chroma. As described above, S304 may be omitted, and one of S302 and S303 may also be omitted.

4.4 Modifications of the Highlighting Processes

As described above, the structure highlighting process and the color highlighting process allow the mucosa layer, the muscle layer, and the fat layer to be displayed in a more easily identifiable manner than before the highlighting processes. However, if performed inappropriately, the highlighting processes may render unnatural colors or may increase noise.

For example, the second image is the G2 image obtained by emission of G2, and G2 has a peak wavelength within the wavelength band intended for identification between the muscle layer and the fat layer. This means that the process of highlighting the structural component of the second image is intended to highlight the difference between the muscle layer and the fat layer. Thus, the region capturing the mucosa layer has a low need for the image processing for identification between the muscle layer and the fat layer, and such image processing may even change the colors, increase noise, or cause other disadvantages.

Hence, the image processing section 17 may detect the mucosa region corresponding to the biological mucosa based on the first image, and may omit a process of highlighting the structural component of the second image for that region determined as the mucosa region. For example, when adding the structural component extracted from the second image, the structure highlighting processing section 17a may exclude the mucosa region from its target regions. Alternatively, when extracting the structural component from the second image, the structure highlighting processing section 17a may exclude the mucosa region from its extraction target regions. This modification can avoid a highlighting process of lower necessity.

One reason for using the first image for detection of the mucosa region is because the first image is captured by emission of the first light (B2) whose wavelength band is set based on the absorption characteristic of the mucosa layer. In other words, use of the first image, which contains information about the mucosa layer, allows for accurate detection of the mucosa region. For example, the image processing section 17 determines pixel values of the first image and detects a region with pixel values at or below a given threshold as the mucosa region. However, the process for detecting the mucosa region is not limited to this. For example, similarly to the aforementioned color determination process, the image processing section 17 may convert the R, G, and B output signals into YCrCb and detect the mucosa region based on information after the conversion. Also in this case, the mucosa region can be detected properly because the B2 image is included in at least one of the R, G, and B signals.

Additionally, the first color highlighting process of highlighting the chroma of the yellow region is intended to increase the difference in chroma between the muscle layer and the fat layer so as to facilitate identification between these layers. Thus, for the region capturing the mucosa layer, the chroma highlighting on the yellow region is disadvantageous and less necessary.

Hence, the image processing section 17 may detect the mucosa region corresponding to the biological mucosa based on the first image, and may omit the first color highlighting process for that region determined as the mucosa region. This modification can also avoid a highlighting process of lower necessity.

Although the embodiments and the modifications thereof have been described in detail above, the present disclosure is not limited to the above embodiments and the modifications thereof, and various modifications and variations may be made without departing from the scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An endoscope apparatus comprising:
an illumination device configured to emit a plurality of illumination lights including first light and second light;
an imaging device configured to capture an image of return light from a subject based on emission by the illumination device; and
a processor including hardware,
wherein the illumination device is configured to:
emit the first light having a peak wavelength within a first wavelength range including a wavelength at which absorbance of a biological mucosa reaches a largest value; and
emit the second light having a peak wavelength within a second wavelength range including a wavelength at which absorbance of a muscle layer reaches a maximum value, absorbance of the second light by fat being lower than absorbance of the second light by the muscle layer,
wherein the processor is configured to:
generate a display image based on a first image that is captured by the imaging device and that corresponds to the first light to thereby display the biological mucosa and the muscle layer in an identifiable manner; and generate the display image based on a second image that is captured by the imaging device and that corresponds to the second light to thereby display the muscle layer and the fat in an identifiable manner.

2. The endoscope apparatus as defined in claim 1, wherein
the first wavelength range including the peak wavelength of the first light is 415 nm±20 nm.

3. The endoscope apparatus as defined in claim 2, wherein
the first light is narrowband light whose wavelength band is narrower than a wavelength band of light used for generation of a white light image.

4. The endoscope apparatus as defined in claim 1, wherein
the second wavelength range including the peak wavelength of the second light is 540 nm±10 nm.

5. The endoscope apparatus as defined in claim 4, wherein
the second light is narrowband light whose wavelength band is narrower than a wavelength band of light used for generation of a white light image.

6. The endoscope apparatus as defined in claim 1, wherein
the illumination device is configured to emit third light, the third light being within a wavelength band in which absorbance of the third light by the biological mucosa is lower than absorbance of the first light by the biological mucosa and in which absorbance of the third light by the muscle layer is lower than absorbance of the second light by the muscle layer.

7. The endoscope apparatus as defined in claim 1, wherein
the processor performs at least one of a process of highlighting a structural component of the first image and a process of highlighting a structural component of the second image.

8. The endoscope apparatus as defined in claim 6, wherein
the processor performs at least one of a process of correcting the first image based on a third image that is captured by the imaging device and that corresponds to the third light and highlighting a structural component of the corrected first image, and a process of correcting the second image based on the third image and highlighting a structural component of the corrected second image.

9. The endoscope apparatus as defined in claim 7, wherein
the processor performs a process of combining a signal corresponding to the structural component into a luminance component in an output.

10. The endoscope apparatus as defined in claim 7, wherein
the processor performs a process of combining a signal corresponding to the structural component into at least one of an R output signal, a G output signal, and a B output signal.

11. The endoscope apparatus as defined in claim 6, wherein
the processor performs a process of highlighting color information based on the first image, the second image, and a third image that is captured by the imaging device and that corresponds to the third light.

12. The endoscope apparatus as defined in claim 11, wherein
based on the first image, the second image, and the third image, the processor performs at least one of a first color highlighting process and a second color highlighting process, the first color highlighting process highlighting chroma of a region that is determined as a yellow region, the second color highlighting process highlighting chroma of a region that is determined as a red region.

13. The endoscope apparatus as defined in claim 11, wherein
based on the first image, the second image, and the third image, the processor performs a third color highlighting process of reducing chroma of a region where the chroma is determined to be below a predetermined threshold.

14. The endoscope apparatus as defined in claim 7, wherein
the processor performs a process of detecting a mucosa region corresponding to the biological mucosa based on the first image, and
the processor does not perform the process of highlighting the structural component of the second image on a region determined as the mucosa region.

15. The endoscope apparatus as defined in claim 12, wherein
the processor performs a process of detecting a mucosa region corresponding to the biological mucosa based on the first image, and
the processor does not perform the first color highlighting process on a region determined as the mucosa region.

16. An operation method of an endoscope apparatus, the method comprising:
emitting a plurality of illumination lights including first light and second light, the first light having a peak wavelength within a first wavelength range including a wavelength at which absorbance of a biological mucosa reaches a largest value, the second light having a peak wavelength within a second wavelength range including a wavelength at which absorbance of a muscle layer reaches a maximum value, absorbance of the second light by fat being lower than absorbance of the second light by the muscle layer;
capturing an image of return light from a subject based on emission of the plurality of illumination lights; and
performing image processing using a captured first image and a captured second image respectively corresponding to the first light and the second light,
wherein, in the image processing, the method further comprises:
generating a display image based on the first image to thereby display the biological mucosa and the muscle layer in an identifiable manner; and generating the display image based on the second image to thereby display the muscle layer and the fat in an identifiable manner.

17. A non-transitory information storage media storing a program, the program causing a computer to execute steps comprising:
causing an illumination device to emit a plurality of illumination lights including first light and second light, the first light having a peak wavelength within a first wavelength range including a wavelength at which absorbance of a biological mucosa reaches a largest value, the second light having a peak wavelength within a second wavelength range including a wavelength at which absorbance of a muscle layer reaches a maximum value, absorbance of the second light by fat being lower than absorbance of the second light by the muscle layer;
capturing an image of return light from a subject based on emission by the illumination device; and
performing image processing using a captured first image and a captured second image respectively corresponding to the first light and the second light,
wherein, at the step of the image processing, the program causes the computer to perform a process of generating a display image based on the first image to thereby display the biological mucosa and the muscle layer in an identifiable manner, and generating the display image based on the second image to thereby display the muscle layer and the fat in an identifiable manner.

18. The endoscope apparatus as defined in claim 1, wherein
the subject is a bladder wall.
